(19) United States
(12) Patent Application Publication
(10) Pub. No.: US 2002/0064800 A1
(43) Pub. Date: May 30, 2002

(54) Microchip

(76) Inventors: Yasuhiro Sando, Amagasaki-Shi (JP); Yasuhisa Fujii, Kyoto-Shi (JP)

Correspondence Address: SIDLEY AUSTIN BROWN & WOOD LLP, 717 NORTH HARWOOD, SUITE 3400, DALLAS, TX, 75201, US

(21) Appl. No.: 09/988,488

(22) Filed: Nov. 20, 2001

(30) Foreign Application Priority Data
Nov. 22, 2000 (JP) 2000-355960
Aug. 20, 2001 (JP) 2001-248884

(51) Int. Cl. G01N 33/53; C12M 3/00; C12M 1/34; G01J 3/00

(52) U.S. Cl. 435/7.1; 435/287.2; 356/300

(57) ABSTRACT

A microchip is disclosed. One embodiment of the microchip has a flow pass for containing a reaction. A detection target region wherein light from the reaction is to be generated is located within at least a portion of the flow pass. The microchip has an optical path for detecting the light from the reaction. A length of the optical path is greater than a depth of the flow pass.

### RELATED APPLICATIONS

This application is based on Patent Application Nos. 2000-855960 and 2001-248884 filed in Japan on Nov. 22, 2000 and Aug. 20, 2001, respectively, the entire contents of which are hereby incorporated by reference.

### FIELD OF THE INVENTION

The present invention relates to a microchip. One embodiment of the present invention relates to a microchip for detecting light when a specimen and reagent are reacted within the microchip. One embodiment of the microchip is suitable for use in blood clot examination, immunological examination, biochemical examination, genetic examination and the like.

### BACKGROUND OF THE INVENTION

In current clinical examinations, an antigen antibody reaction or an enzyme reaction can be used in an immunological examination or a biochemical examination to detect a target material. The detection is mainly accomplished optically, including methods for detecting fluorescence generated by excitation light, and methods for detecting the turbidity of a liquid (nephelometry). The detection is generally captured by a photodiode, photomultiplier and the like, using an excitation light such as that produced by a laser, LED, halogen lamp or the like. For example, when fluorescein is used in a marker, a measurement wavelength near 515 nm is used relative to an excitation light wavelength of 495 nm.

For example, when an antigen antibody reaction is detected by fluorescence, an antibody A anchors to the vessel as shown in FIG. 10(a), an antibody B in the specimen bonds with the antibody A, as shown in FIG. 10(b), and forms a complex (in a primary reaction). After the unreacted liquid is removed, a marker antibody C is added. The marker antibody C bonds with the complex of the bonded antibody B and antibody A to form another complex (in a secondary reaction), as shown in FIG. 10(c). After the unreacted liquid is removed, a substrate fluid (substrate D) including HPPA (p-hydroxyphenylpropionic acid) is added, and a fluorescent material E is generated (in an enzyme reaction) by bonded peroxidase (POD), as shown in FIG. 10(d). Then, the fluorescent material E is irradiated by, for example, excitation light of 323 nm wavelength, and the POD can be quantified with high sensitivity by measuring the generated fluorescence (detection light) at 410 nm wavelength.

In conventional large-scale and intermediate-scale reaction detection devices, such as shown in FIG. 9, for example, a cuvette 4 contains a reaction liquid 5 to which has been added a marker antibody (which generates light and generates fluorescence) used in an antigen antibody reaction for immunological examination. The cuvette 4 is irradiated by excitation light 2a from a light source 2, so as to generate fluorescence 5a. The fluourescence 51, and the light emitted by the reaction liquid 5 itself, is detected by a light-detecting unit 6. In the case of biochemical examination, a calorimetric method or nephelometric method is used. In clotting examination, scattered light detection is generally used.

For example, an immunological examination device such as a model AIA-600II (from Tosoh Corporation, Tokyo, Japan) detects fluorescence from a marker antibody used in an antigen antibody reaction. The device requires large amounts of specimen and reagents. Furthermore, blood clotting examination devices such as a model CA-7000 (from Sysmex Corporation, Kobe, Japan) detect blood clots by detecting a change in scattered light as a result of incident light. In these devices, a cuvette is used with large amounts of specimen and reagents.

Recently, attention has focused on μ-TAS (μ-total analysis system) for miniaturizing devices for use in various processes, such as chemical analysis and synthesis and the like, and for its application to micro-machine technology. Advantages of miniaturized μ-TAS include the use of small amounts of specimen, a short reaction time, and less waste product as compared to conventional devices. Furthermore, when applied to the field of health care, it is expected to reduce the burden on patients by using a small amount of specimen, and to lower the cost of examination by reducing the amount of reagent used. Furthermore, the examination is made more efficient as a result of the greatly reduced reaction time due to the small quantities of specimen and reagent used. These advantages are extremely valuable when applied to immunological examination, biochemical examination, genetic examination and the like. Since the amounts of specimen and reagents are reduced, this method is also applicable to blood clot examination.

For example, when using the previously mentioned devices to detect fluorescence from the antigen antibody reaction, the quantity of specimen is large and the quantity of light to be detected is large. However, when the amount of specimen is reduced, the amount of light to be detected is also reduced, and it becomes difficult to detect the reaction. When a reaction is detected within a fine flow pass, as in a microchip, there is insufficient detection light due to the small amount of specimen, such that detection sensitivity is reduced. Although detection sensitivity can be increased if a large device such as a photomultiplier, cooled CCD or the like is used, the reaction detection device becomes large-scale thereby, and also becomes expensive.

### SUMMARY OF THE INVENTION

Accordingly, one embodiment of the present invention aims to eliminate these problems of the art by providing a microchip capable of miniaturizing a reaction detection device using the microchip.

To eliminate the previously mentioned problems of the art, one embodiment of the present invention provides a microchip having the structure described below.

The microchip is a type having a fine flow pass for reacting specimen and reagent. The microchip is capable of letting light exit. The light is generated within a detection target region of the flow pass, and exits to a specific position outside the microchip. The length of the optical path within the detection target region, or the length of the detection target region itself, is greater than the width and the depth of the flow pass.

According to this structure, in the detection target region (i.e., the region of light detection), the length of the optical path within this region, or the length of the region itself, is greater than the width and the depth of the flow pass, such that weak light produced by the reaction of specimen and reagent within the flow pass can be efficiently detected. It is not necessary to provide a light collection unit or a large-scale detection device to increase sensitivity in the reaction detection device to detect weak light as is required in conventional devices.

Accordingly, the reaction detection device using the microchip can be compact.

Specifically, the microchip can be structured in various embodiments as described below.

In accordance with one embodiment, detection light exits the microchip at one end of the detection target region in an extension direction of the flow pass. The detection light is generated within the detection target region of the flow pass. According to this structure, light can be accumulated throughout the detection target region and can be detected since the length of the flow pass in the extension direction, and the detection target region itself, can be longer than when detecting only light exiting from a part of the flow pass in a direction perpendicular to the extension direction of the flow pass (i.e., a depth direction). Specifically, for example, a layer having a suitable refractive index is formed along the flow pass, such that light advancing in a direction towards the outside of the flow pass is reflected so as to return the light to within the flow pass.

In accordance with another embodiment, it is desirable that a reflective film be formed on the surface forming the flow pass. According to this structure, since light advancing in a direction towards the outside of the flow pass is reflected so as to return the light to within the flow pass. When viewed in total, the light generated within the flow pass advances along the flow pass and is compiled. The reflective film efficiently compiles the light.

In accordance with one embodiment, it is desirable that the microchip is provided with a light guide unit. One end of the light guide unit is disposed adjacent to at least one end of the detection target region of the flow pass. In one embodiment wherein the flow pass is curved, the light guide unit extends from that one end of the detection target region largely in a tangential direction to the flow pass. The other end of the light guide unit is exposed outside the microchip. Light can pass between the one end and the other end of the light guide unit. According to this structure, for example, light generated in the detection target region of the flow pass may exit to the outside of the microchip via the light guide unit, or excitation light may enter the detection target region of the flow pass through the light guide unit from outside the microchip. For example, a simple structure is possible wherein light may enter or exit from an intermediate part of the flow pass (e.g., the detection target region) even if the end of the flow pass is bent or curved for introducing specimen and reagent to the flow pass, or for removing air therefrom. The light guide unit may be structured so as to have different refractive indices at the center part and at the circumference as, for example, an optical fiber.

One embodiment of the microchip may allow light generated from within the detection target region of the flow pass to exit in an extension direction and in a direction perpendicular to the flow pass.

In accordance with one embodiment, it is desirable that the microchip comprise a lens having optical power in a direction perpendicular to the extension direction of the flow pass. According to this structure, light generated within the detection target region of the flow pass is condensed by the lens and exits to the outside of the microchip.

### BRIEF DESCRIPTION OF THE DRAWINGS

These and other objects, advantages and features of the invention will become readily apparent from the following Detailed Description of the Preferred Embodiments taken in conjunction with the accompanying drawings. Throughout the accompanying drawings, like parts are designated by like reference numbers, and in which:

FIG. 1 is a plan view of a microchip;

FIG. 2 is a cross section view of the microchip of FIG. 1;

FIGS. 3(a) and 3(b) show a plan view and a cross section view of a first embodiment of a microchip of the present invention;

FIG. 4 is a cross section view of a second embodiment of a microchip of the present invention;

FIG. 5 is a cross section view of a third embodiment of a microchip of the present invention;

FIG. 6 is a plan view of a first modification of a microchip of the present invention;

FIG. 7 is a plan view of a second modification of a microchip of the present invention;

FIG. 8 is a plan view of a third modification of a microchip of the present invention;

FIG. 9 illustrates a light detection method; and

FIGS. 10(a)-10(d) illustrate a fluorescence detection method in accordance with the present invention.

### DETAILED DESCRIPTION OF THE PREFERRED EMBODIMENTS

A microchip in accordance with each embodiment of the present invention is described below with reference to the accompanying drawings.

First, the basic structure of the microchip is described.

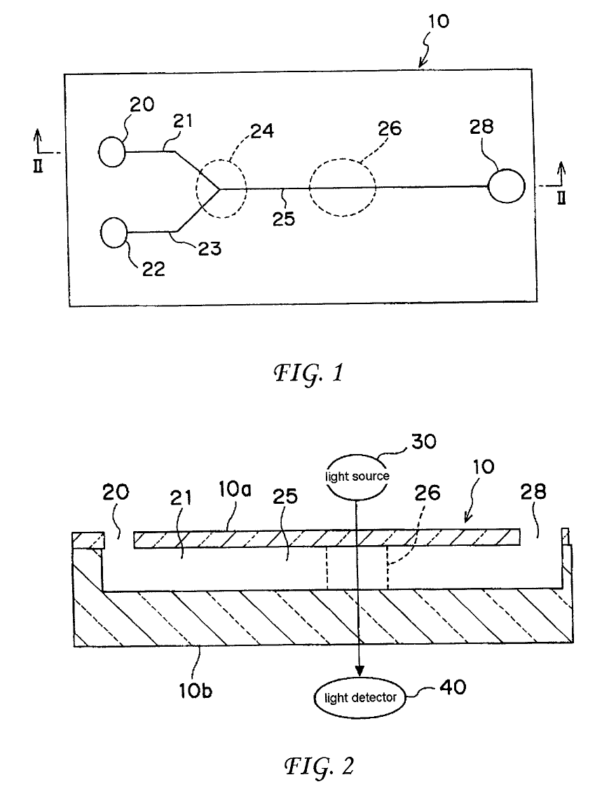

The microchip 10 comprises fine flow passes 21, 23, 25 formed on a substrate 10b and covered by a cover 10a, as shown schematically in the plan view of FIG. 1 and in the cross section view of FIG. 2.

For example, the external dimensions of the microchip 10 are approximately 20×40×0.5 mm. The width of each of the flow passes 21, 23, 25 is 200 μm, and the depth is approximately 100 μm.

Specifically, as shown in FIG. 1, a specimen supply inlet 20 for supplying liquid specimen is provided at one end of the flow pass 21. A reagent supply inlet 22 for supplying liquid reagent is provided at one end of the flow pass 23. An discharge port 28 or ventilator port 28 is provided at one end of the flow pass 25.

Specimen and reagent flow through the flow passes 21 and 23, and join so as to be mixed at a confluence area 24 (indicated by the dotted circle). For example, the specimen and reagent jointly flow in a laminar state within the flow pass 25, which has a narrow width, so as to become mixed by diffusion. After confluence, the confluent specimen and reagent move in the flow pass 25 toward the ventilator port 28, and the reaction of the specimen and reagent is detected.

In order to detect the reaction of the specimen and reagent, the microchip 10 is installed in a reaction detection device (not shown in the drawings). The reaction detection device (a main unit) is provided with a light source 30, such as an LED or the like, on top of an area 26 of the microchip 10. The reaction detection device is provided with a light detector 40, such as a photodiode or the like, on the bottom of the microchip 10, for example, as shown in FIG. 2, such that light from the area 26 is received by the light detector 40. As shown in FIG. 2, the length of an optical path (i.e., the length along which light passes through the reaction liquid) for detecting the reaction within the flow pass 25 is extremely short, approximately 100 μm in this example.

In this case, detection is difficult when there is a small amount of specimen. The reaction is weak, and the intensity of the light to be detected is very weak. The microchip of each embodiment of the present invention has the structure described below. Hereinafter, the description focuses on points of departure from the basic structure shown in FIGS. 1 and 2; like parts are designated by like reference numbers in the drawings.

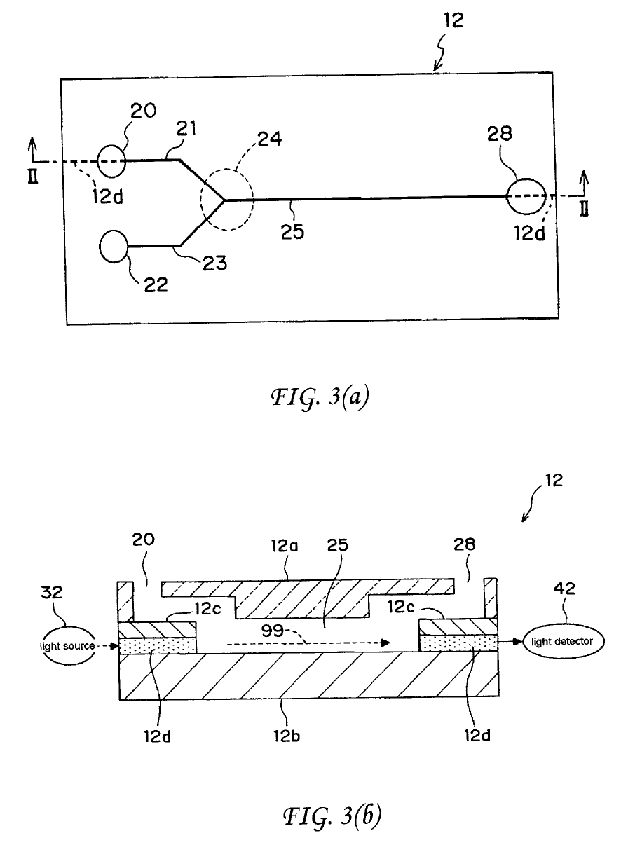

FIGS. 3(a) and 3(b) show a first embodiment of a microchip 12 of the present invention. The microchip 12 has an optical path oriented in a direction of extension of the flow pass 25, i.e., in the direction of flow (as indicated by the arrow 99). The entirety of the flow pass 25 is a detection target region, as shown in the plan view and cross section view of FIGS. 3(a) and 3(b). Since the length of the flow pass 25 in the direction of flow 99 is approximately 20 mm, the length of the optical path is approximately 200 times that shown in FIG. 2, and the amount of detectable light is greatly increased.

A light guide unit, such as an optical waveguide, is used to transmit light to the flow pass 25. The optical waveguide has a core area 12d of SiO₂, and a clad area 12c of germanium or fluoride doped SiO₂. This arrangement is advantageous in that, since SiO₂ is hydrophilic, it is easy to load a liquid in the microchip 12.

The optical waveguide can be built in a batch together with the flow pass 25 and the like on the microchip 12 by a micro-machining process. That is, the core area 12d can be formed by a SiO₂ patterning (spatter) process, which forms the optical waveguide on a silicon substrate 12b. A film of germanium or fluoride doped SiO₂ can be placed thereupon to form the clad area 12c. These areas are patterned to form the flow pass 25. For example, this patterning may be accomplished by RIE (reactive ion etching), which is a dry etching method for anisotropic dry etching by ions on a substrate at high speed, or by RIE followed by ICP (inductively coupled plasma), which is an anisotropic dry etching method capable of deep channel processing. Finally, a glass cover 12a is cemented on the silicon substrate 12b.

A reaction detection device (not shown) in which the microchip 12 is installed is provided with a light source 32 for irradiating light within the flow pass 25 from a first portion of the core area 12d of the optical waveguide. The reaction detection device is provided with a light detector 42 for receiving light exiting through a second portion of the core area 12d of the optical waveguide, as shown in FIG. 3(b). An LED, a laser such as Ar laser, or the like is used as the light source 32. A photodiode or the like is used as the light detector 42.

Polyimide may be used in the optical waveguide. A resin such as PMMA (polymethyl methacrylate), silicon or the like may be used in the cover 12a. Optical fiber also may be embedded in the microchip 12 rather than using the optical waveguide.

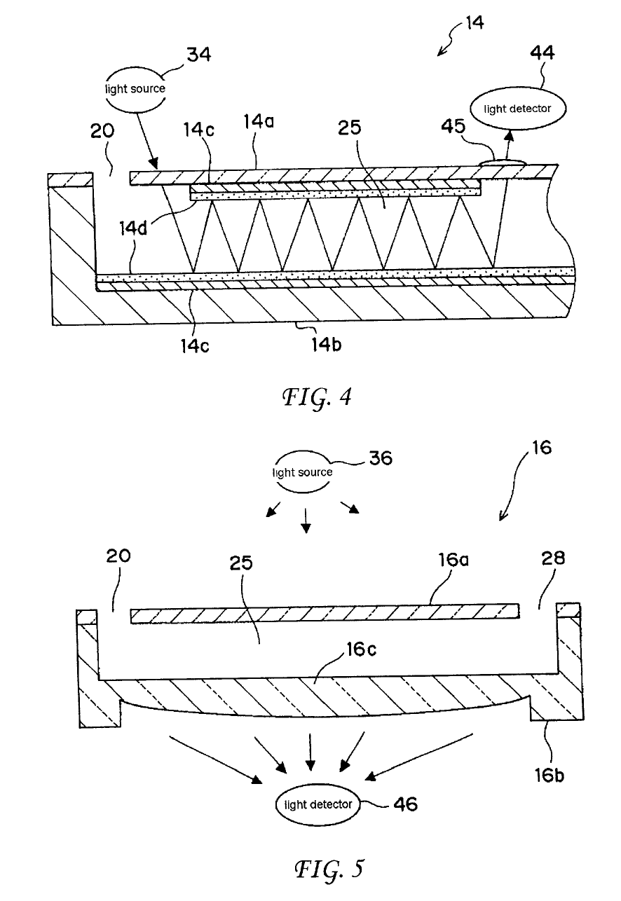

FIG. 4 shows a cross section of a second embodiment of a microchip 14 in accordance with the present invention.

The microchip 14 has reflection-enhancing mirror films 14c formed on the top and bottom surfaces of the flow pass 25, such that light is reflected in vertical directions within the flow pass 25 while advancing in the direction of extension of the flow pass 25, thereby increasing the length of the optical path. The mirror films 14c are formed on cover 14a and substrate 14b of the microchip 14 by spattering, vacuum deposition or the like using a metallic film (Ag, Au, Al or the like). A protective film 14d of SiO₂ is formed thereupon. This arrangement is advantageous in that, since SiO₂ is hydrophilic, it is easy to load a liquid in the microchip 14.

A reaction detection device (not shown) in which the microchip 14 is installed is provided with a light source 34 for directing light from one end of the flow pass 25 of the microchip 14, and a light detector 44 for receiving light exiting from the other end of the flow pass 25. An LED, laser such as Ar laser, or the like is used as the light source 34. A photodiode or the like is used as the light detector 44.

Light entering the flow pass 25 of the microchip 14 from the light source 34 is reflected by the reflective films 14c as the light advances to the light detector 44. The length of the optical path increases in accordance with the number of reflections, thereby extending the detectable limits of the reflected light. For example, if the number of reflections is 200, the length of the optical path is increased 200 times.

The microchip 14 has a lens 45 disposed adjacent to an end of the flow pass 25 proximate the light detector 44. The lens 45 condenses light exiting the flow pass 25, and directs the light to the light detector 44. Since the lens 45 is provided on the microchip 14, a component for condensing light need not be provided on the reaction detection device side.

FIG. 5 shows a cross section of a microchip 16 of a third embodiment of the present invention.

The microchip 16 is provided with a condensing lens unit 16c having a processed convex lens shape on a back end part (substrate 16b side) of the flow pass 25, so as to condense light from the flow pass 25 (approximately 20 mm). The number of components is not increased because the substrate 16b of the microchip 16 is formed of a resin such as PMMA, PDMS (polydimethyl siloxane) or the like, so as to integratedly include the condensing lens unit 16c.

A reaction detection device (not shown) in which the microchip 16 is installed is provided with a light source 36, and a light detector 46. The light source 36 is disposed on the cover 16a side of the microchip 16, so as to irradiate excitation light in the flow pass 25 of the microchip 16. The light detector 46 is disposed opposite the condensing lens unit 16c of the substrate 16b, so as to receive light from the flow pass 25, which has been condensed by the condensing lens unit 16c. Since the condensing lens unit 16c is integratedly formed from the microchip 16, components for condensing light need not be provided on the reaction detection device side.

The condensing lens unit 16c of FIG. 5 is formed in a direction of flow. In another embodiment, a condensing lens unit is formed in a cross-flow direction of the flow pass 25 and a line-type photodetector is disposed as a light detector, so as to enable a change in the direction of the reaction flow in the flow pass 25 (time axis change) to be detected.

The embodiments of the microchips 12, 14, 16 described above increase detection sensitivity by condensing low detectable light, which is due to the small amount of specimen, from the detection target region of the flow pass 25, and by increasing the length of the detection optical path. Since the structures for increasing detection sensitivity are incorporated in the microchips 12, 14, and 16, a reaction detection device using one of the microchips 12, 14, 16 to detect a reaction need not be a large-scale device, and can be inexpensive.

The specimen (blood) and reagent may be used in extremely minute amounts since the reaction is detected within the fine flow pass 25. Since an extremely minute amount of liquid is used, a reaction occurs quickly, and detection time is extremely short, thereby providing greater detection efficiency, which is advantageous particularly when speed is required, as in an emergency.

Since the reaction detection device can be compact, such a device is suitable for use at a POC (point of care), and may be used for examination within the home, and in an ambulance when speed is required.

The present invention is not limited to the previously described embodiments, and various other modifications thereto are possible.

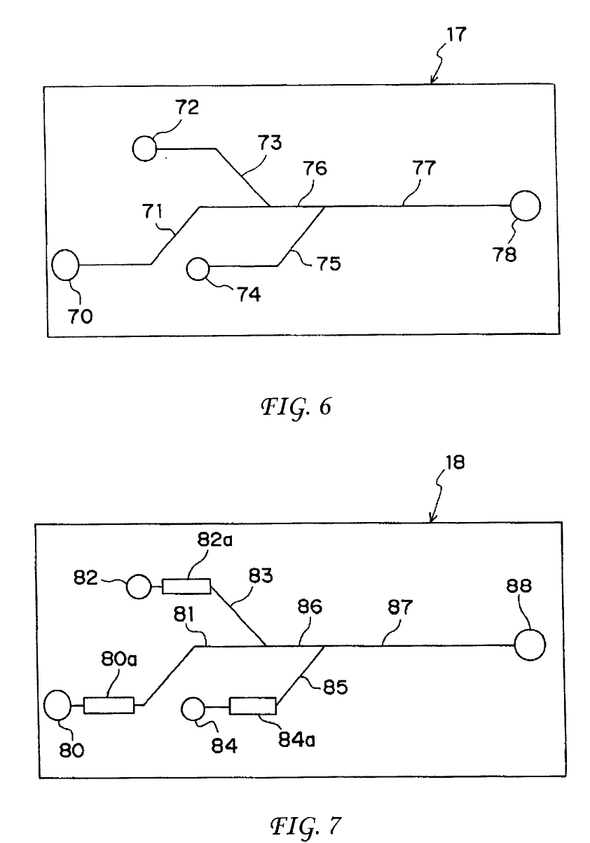

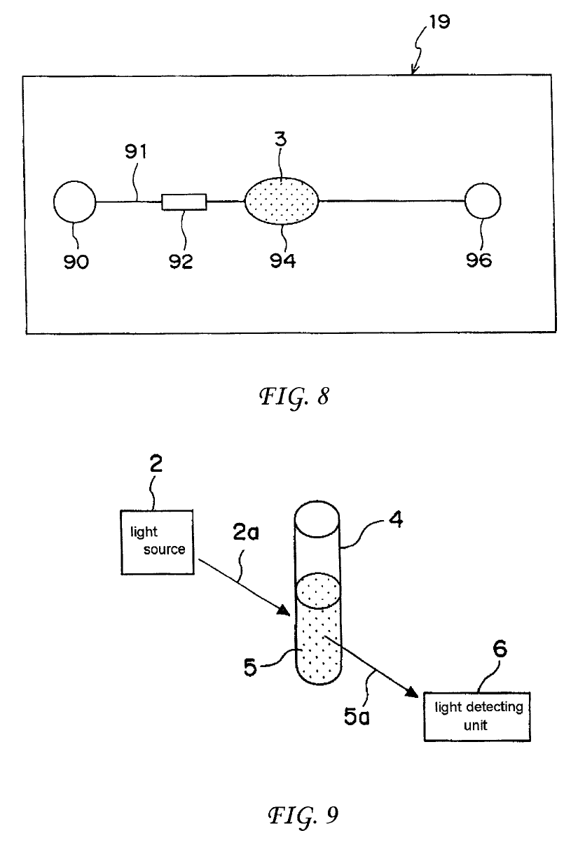

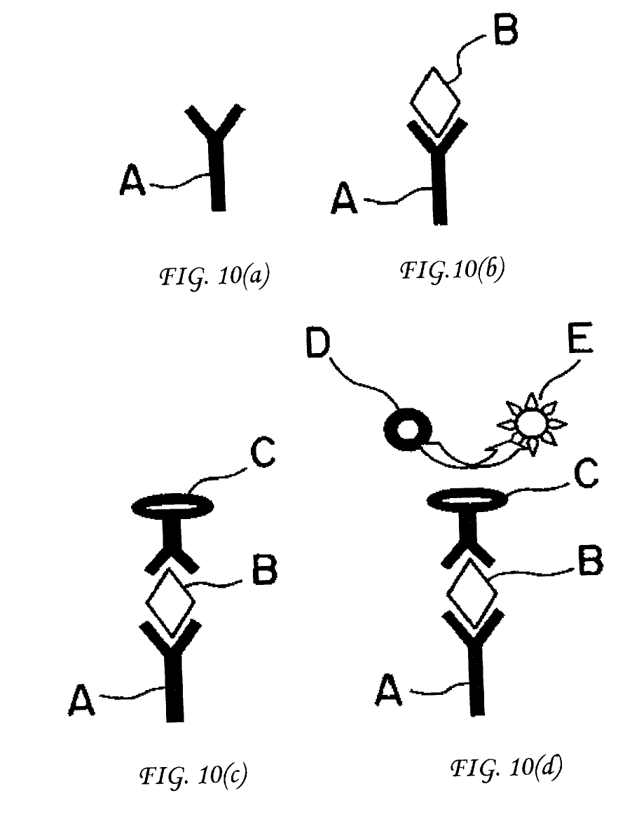

For example, a plurality of specimens and/or reagents may be used. FIG. 6 shows a microchip 17 of a first modification of the present invention wherein two reagents are used. As shown in FIG. 6, a specimen supply inlet 70, and two reagent supply inlets 72 and 74 may be provided, such that the specimen and reagents flow along flow passes 71, 73, 75, 76, 77 toward a discharge port 78. The specimen and reagents are mixed in flow passes 76 and 77, and one or more reactions occur. In this case it is desirable that the flow pass 77 in which the final mixed liquid flows is the detection target region.

FIG. 7 shows a microchip 18 of a second modification of the present invention. As shown in FIG. 7, micro pumps 80a, 82a, 84a may be provided in flow passes 81, 83, 85, such that specimen and reagents supplied from inlets 80, 82, 84 flow toward the discharge port 88, so as to join and be mixed in flow passes 86 and 87, and one or more reactions occur. In this case it is desirable that the flow pass 87 is the detection target region.

Furthermore, the specimen need not be a liquid. For example, a solid specimen may be anchored within the flow pass.

FIG. 8 shows a microchip 19 of a third modification of the present invention. For example, a reagent fixing unit 94 may be provided at a suitable position in the flow pass 91 between a specimen supply inlet 90 and a ventilator port 96. A solid reagent 3 may be temporarily anchored in the reagent fixing unit 94 beforehand. When the reagent 3, which is temporarily anchored in the reagent fixing unit 94, makes contact with a specimen fed by a micro pump 92, for example, the reagent 3 is peeled from the reagent fixing unit 94 or is dissolved so as to mix with the specimen. In this case, it is desirable that the region in the flow pass 91 between the reagent fixing unit 94 and the ventilator port 96 is the detection target region.

The detection need not be of light generated by fluorescence, and may be of light generated by electrochemical generation. In this case a light source for excitation light is unnecessary, and an electrode must be provided along the flow pass of the microchip.

Although the present invention has been fully described by way of examples with reference to the accompanying drawings, it is to be noted that various changes and modifications will be apparent to those skilled in the art. Therefore, unless otherwise such changes and modifications depart from the scope of the present invention, they should be construed as being included therein.

What is claimed is:

1. A microchip, comprising:
a flow pass for containing a reaction;
a detection target region wherein light from said reaction is to be generated, said detection target region being located within at least a portion of said flow pass; and
an optical path, at least a part of said optical path being located within said detection target region, for detecting said light from said reaction, said light exiting to a light detection area;
wherein a length of said part of said optical path is greater than a depth of said flow pass.

2. A microchip in accordance with claim 1, wherein said light is generated in response to excitation light from a light source.

3. A microchip in accordance with claim 1, wherein said length of said part of said optical path is greater than a width of said flow pass.

4. A microchip, comprising:
a flow pass for containing a reaction; and
a detection target region, located within at least a portion of said flow pass, wherein light from said reaction is generated, said light passing through said detection target region to a light detection area;
wherein said detection target region is larger than said light detection area.

5. A microchip in accordance with claim 4, wherein said light is generated in response to excitation light from a light source.

6. A microchip, comprising:
a flow pass for containing a reaction;
a detection target region wherein light from said reaction is to be generated, said detection target region being located within at least a portion of said flow pass; and
an optical path for detecting said reaction, said optical path oriented in an extension direction of said flow pass.

7. A microchip in accordance with claim 6, wherein said light is generated in response to excitation light from a light source.

8. A microchip in accordance with claim 6, wherein a length of said detection target region is greater than a depth and a width of said flow pass.

9. A microchip in accordance with claim 6 further comprising:
a plurality of supply inlets for supplying a plurality of fluids; and
a plurality of branch flow passes, respectively connecting said plurality of supply units to said flow pass.

10. A microchip in accordance with claim 9 further comprising a plurality of micro pumps respectively disposed in said plurality of branch flow passes for pumping said fluids into said flow pass.

11. A microchip in accordance with claim 9 further comprising an area in said flow pass for anchoring a solid specimen.

12. A microchip in accordance with claim 6 further comprising a reagent fixing unit located in said flow pass.

13. A microchip in accordance with claim 6, wherein said optical path comprises a light guide unit, disposed adjacent to an end of said detection target region, for conducting said light between said detection target region and a light detection area.

14. A microchip in accordance with claim 13, wherein said light guide unit comprises an optical fiber.

15. A microchip in accordance with claim 13, wherein said optical path further comprises a second light guide unit connected to a second end of said detection target region.

16. A microchip in accordance with claim 13, wherein said light guide unit comprises an optical waveguide.

17. A microchip, comprising:
a flow pass for containing a reaction;
a detection target region wherein light from said reaction is to be generated, said detection target region being located within at least a portion of said flow pass;
a reflective surface formed on at least a portion of a surface of said detection target region; and
an optical path for detecting said reaction;
wherein said reflective surface is adapted to reflect said light so as to increase a length of said optical path beyond a length of said detection target region.

18. A microchip in accordance with claim 17, wherein said light is generated in response to excitation light from a light source.

19. A microchip in accordance with claim 17, wherein a length of said optical path is greater than a depth and a width of said flow pass.

20. A microchip in accordance with claim 17, wherein said reflective surface is formed on a top surface and a bottom surface of said detection target region of said flow pass.

21. A microchip in accordance with claim 17, wherein said reflective surface comprises a metallic film.

22. A microchip in accordance with claim 17, further comprising a lens disposed adjacent to an end of said detection target region of said flow pass.

23. A microchip in accordance with claim 22, wherein said lens is adapted to condense said light as said light exits said detection target region of said flow pass and to direct said light to a light detection area.

24. A microchip in accordance with claim 17 further comprising:
a plurality of supply inlets for supplying a plurality of fluids; and
a plurality of branch flow passes, respectively connecting said plurality of supply units to said flow pass.

25. A microchip in accordance with claim 24 further comprising a plurality of micro pumps respectively disposed in said plurality of branch flow passes for pumping said fluids into said flow pass.

26. A microchip in accordance with claim 24 further comprising an area in said flow pass for anchoring a solid specimen.

27. A microchip in accordance with claim 17 further comprising a reagent fixing unit located in said flow pass.

28. A microchip, comprising:
a substrate;
a flow pass for containing a reaction, said flow pass formed on a first side of said substrate;
a detection target region wherein light from said reaction is to be generated, said detection target region being located within at least a portion of said flow pass;
a condensing lens unit for condensing said light, said condensing lens unit formed on a second side of said substrate.

29. A microchip in accordance with claim 28, wherein said light is generated in response to excitation light from a light source.

30. A microchip in accordance with claim 28, wherein said condensing lens unit comprises a convex lens.

31. A microchip in accordance with claim 28, wherein said condensing lens unit possesses optical power in a direction perpendicular to an extension direction of said flow pass.

32. A microchip in accordance with claim 28, wherein said condensing lens unit has a curvature in an extension direction of said flow pass.

33. A microchip in accordance with claim 28, wherein said condensing lens unit has a curvature in a cross-flow direction of said flow pass.

34. A microchip in accordance with claim 28 further comprising:
a plurality of supply inlets for supplying a plurality of fluids; and
a plurality of branch flow passes, respectively connecting said plurality of supply units to said flow pass.

35. A microchip in accordance with claim 34 further comprising a plurality of micro pumps respectively disposed in said plurality of branch flow passes for pumping said fluids into said flow pass.

36. A microchip in accordance with claim 34 further comprising an area in said flow pass for anchoring a solid specimen.

37. A microchip in accordance with claim 34 further comprising a reagent fixing unit located in said flow pass.

38. A method of manufacturing a microchip, comprising the steps of:
providing a substrate;
forming a core area of an optical waveguide on said substrate;
placing a film on said core area to form a clad area;
patterning a portion of said core area and said clad area to form an portion of a flow pass therein; and
placing a cover over said substrate.

39. A method in accordance with claim 38, wherein said step of forming said core area comprises a SiO2 patterning process.

40. A method in accordance with claim 38, wherein step of patterning comprises an anisotropic dry etching process.

41. A method of manufacturing a microchip, comprising the steps of:
providing a substrate;
forming a reflective film on said substrate;
forming a protective film on said reflective film on said substrate;
providing a cover;
forming a reflective film on said cover;
forming a protective film on said reflective film on said cover; and
placing said cover over said substrate.

42. A method in accordance with claim 41, wherein said reflective film comprises a metallic material.

43. A method of manufacturing a microchip, comprising the steps of:
providing a substrate;
forming a condensing lens on one side of said substrate;
forming a flow pass on a second side of said substrate; and
placing a cover over said substrate proximate said flow pass.

44. A method in accordance with claim 43, wherein said condensing lens has a curvature in an extension direction of said flow pass.

45. A method in accordance with claim 43, wherein said condensing lens has a curvature in a direction perpendicular to an extension of said flow pass.
